(19) United States
(12) Patent Application Publication
(10) Pub. No.: US 2024/0082365 A1
(43) Pub. Date: Mar. 14, 2024

(54) MULTIDISCIPLINARY TREATMENT OF CHRONIC DISEASE USING REGENERATIVE CELLS AND TECHNOLOGIES

(71) Applicant: James VELTMEYER, La Jolla, CA (US)

(72) Inventor: James VELTMEYER, La Jolla, CA (US)

(21) Appl. No.: 18/464,462

(22) Filed: Sep. 11, 2023

Related U.S. Application Data
(60) Provisional application No. 63/405,411, filed on Sep. 10, 2022.

(51) Int. Cl. A61K 38/44; A61K 31/015; A61K 31/355; A61K 31/375; A61K 31/4706; A61K 33/04; A61K 33/30; A61K 38/06; C12N 15/86

(52) U.S. Cl. CPC A61K 38/446; A61K 31/015; A61K 31/355; A61K 31/375; A61K 31/4706; A61K 33/04; A61K 33/30; A61K 38/063; C12N 15/86

(57) ABSTRACT

The invention provides assessment means, treatment protocols and compositions of matter for the personalized therapy of chronic and/or degenerative disease. In one embodiment a patient is assessed for various body and disease parameters and a multistep approach is undertaken which addresses: a) hormonal abnormalities; b) genetic composition; c) immunological status; d) nutrient deficiencies; e) oxidative stress; and f) systemic cytokine abnormalities. Subsequent and/or concurrent with addressing abnormalities various therapeutic interventions are utilized including immunotherapy, extracorporeal pheresis, hormonal and metabolic therapy. Numerous combinations of regenerative cells are described as well as combinations useful for treatment and/or prevention of disease.

### CROSS-REFERENCE TO RELATED APPLICATIONS

This application claims priority to U.S. Provisional Application No. 63/405,411, titled “Multidisciplinary Treatment of Chronic Disease Using Regenerative Cells and Technologies”, filed Sep. 10, 2022, which is hereby incorporated by reference in its entirety.

### FIELD OF THE INVENTION

The teachings herein are related to the treatment of chronic disease using regenerative cells and antioxidants and anti-inflammatory agents

### Background

Stem cells act as regenerative cells. The body is known to possess different regenerative compartments within tissues. The most commonly known one is bone marrow which produces approximately 5 billion blood cells per minute. Within the bone marrow stem cells reside in hypoxic niches. When stem cells are taken out of hypoxic areas and oxygen tension is increased, there is a correlative decrease in regenerative potential. In the area of other regenerative tissues, there has been little studies to determine the effects of oxygen tension on regenerative activity. Stem cells offer the possibility of treating numerous disease conditions, unfortunately, with some exceptions, the majority of stem cell studies in the Phase III have not yielded stunning results. The numerous comorbidities associated with chronic disease suppress in the in vivo efficacy of cellular approaches. In the current disclosure means of enhancing stem cell activity for treatment of chronic degenerative conditions are disclosed.

### SUMMARY

Preferred embodiments are drawn to methods of enhancing stem cell activity in the treatment of a degenerative condition though administering at least one or more antioxidants and/or anti-inflammatory agents before, at the same time has, and subsequent to stem cell therapy.

Preferred methods include embodiments wherein said degenerative condition is autoimmune.

Preferred methods include embodiments wherein said degenerative condition is immunologically mediated.

Preferred methods include embodiments wherein said degenerative condition is mediated by injury.

Preferred methods include embodiments wherein said degenerative condition is mediated by genetic predisposition.

Preferred methods include embodiments wherein said antioxidant is super oxide dismutase.

Preferred methods include embodiments wherein said super oxide dismutase is manganese dependent.

Preferred methods include embodiments wherein the gene encoding manganese dependent superoxide dismutase is administered locally into an area proximal to the diseased tissue.

Preferred methods include embodiments wherein said gene encoding manganese dependent superoxide dismutase is transfected into diseased tissue by means of a viral vector.

Preferred methods include embodiments wherein said gene encoding manganese dependent superoxide dismutase is transfected into diseased tissue by means of a hydrodynamic transfection.

Preferred methods include embodiments wherein said gene encoding manganese dependent superoxide dismutase is transfected into diseased tissue by means of a mRNA liposomal delivery.

Preferred methods include embodiments wherein said gene encoding manganese dependent superoxide dismutase is transfected into diseased tissue by means of naked DNA administration.

Preferred methods include embodiments wherein said antioxidant is zinc.

Preferred methods include embodiments wherein said antioxidant is vitamin C.

Preferred methods include embodiments wherein said antioxidant is vitamin E.

Preferred methods include embodiments wherein said antioxidant is selenium.

Preferred methods include embodiments wherein said antioxidant is Trolox.

Preferred methods include embodiments wherein said antioxidant is ebselen.

Preferred methods include embodiments wherein said antioxidant is glutathione.

Preferred methods include embodiments wherein said antioxidant is carotene.

Preferred methods include embodiments wherein said antioxidant is ubiquinol.

Preferred methods include embodiments wherein said antioxidant is propyl gallate.

Preferred methods include embodiments wherein said antioxidant is hydrogen gas.

Preferred methods include embodiments wherein said antioxidant is xenon gas.

Preferred methods include embodiments wherein said antioxidant is argon gas.

Preferred methods include embodiments wherein said antioxidant is neon gas.

Preferred methods include embodiments wherein said antioxidant is krypton gas.

Preferred methods include embodiments wherein said antioxidant is butylated hydroxytoluene.

Preferred methods include embodiments wherein said antioxidant is butylated hydroxyanisole.

Preferred methods include embodiments wherein said antioxidant is butylated hydrogen sulfide.

Preferred methods include embodiments wherein said antioxidant is erythrobate.

Preferred methods include embodiments wherein said antioxidant is sodium tripolyphosphate.

Preferred methods include embodiments wherein said antioxidant is ethylenediaminetetraacetic acid.

Preferred methods include embodiments wherein said antioxidant is ethoxyquin.

Preferred methods include embodiments wherein said antioxidant is casein.

Preferred methods include embodiments wherein said antioxidant is pyruvate.

Preferred methods include embodiments wherein said antioxidant is minocycline.

Preferred methods include embodiments wherein said antioxidant is tetracyclin.

Preferred methods include embodiments wherein said anti-inflammatory agent is hydroxychloroquine.

Preferred methods include embodiments wherein said anti-inflammatory agent is an NF-kappa B inhibitor.

Preferred methods include embodiments wherein said NF-kappa B inhibitor is hydroxychloroquine.

Preferred methods include embodiments wherein said NF-kappa B inhibitor is Calagualine.

Preferred methods include embodiments wherein said NF-kappa B inhibitor is Conophylline.

Preferred methods include embodiments wherein said NF-kappa B inhibitor is Evodiamine.

Preferred methods include embodiments wherein said NF-kappa B inhibitor is Geldanamycin.

Preferred methods include embodiments wherein said NF-kappa B inhibitor is selected from a group comprising of: Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+istonic).

Preferred methods include embodiments wherein an inhibitor of inflammatory cytokines is administered systemically and/or locally in the diseased tissue prior to, concurrent with, or subsequent to stem cell administration.

Preferred methods include embodiments wherein said inflammatory cytokines are selected from a group comprised of: inflammatory cytokines are cytokines capable of inducing expression of genes in endothelial cells selected from a group comprising of: IL-6, Myosin 1, IL-33, Hypoxia Inducible Factor-1, Guanylate Binding Protein Isoform I, Aminolevulinate delta synthase 2, AMP deaminase, IL-17, DNAJ-like 2 protein, Cathepsin L, Transcription factor-20, M31724, pyenylalkylamine binding protein; HEC, GA17, arylsulfatase D gene, arylaulfatase E gene, cyclin protein gene, pro-platelet basic protein gene, PDGFRA, human STS WI-12000, mannosidase, beta A, lysosomal MANBA gene, UBE2D3 gene, Human DNA for Ig gamma heavy-chain, STRL22, BHMT, Homo Sapiens Down syndrome critical region, FI5613 containing ZNF gene family member, IL8, ELFR, Homo Sapiens mRNA for dual specificity phosphatase MKP-5, Homo Sapiens regulator of G protein signaling 10 mRNA complete, Homo sapiens Wnt-13 Mma, Homo Sapiens N-terminal acetyltransferase complex ard1 subunit, ribosomal protein L15 mRNA, PCNA mRNA, ATRM gene exon 21, HR gene for hairless protein exon 2, N-terminal acetyltransferase complex and 1 subunit, HSM801431 Homo Sapiens mRNA, CDNA DKFZp434N2072,RPL26, and HR gene for hairless protein, regulator of G protein signaling.

Preferred methods include embodiments wherein an immune suppressive agent is administered prior to, concurrent with or subsequent to stem cell administration.

Preferred methods include embodiments wherein said immune suppressive agent is cyclophosphamide.

Preferred methods include embodiments wherein said immune suppressive agent is prednisone.

Preferred methods include embodiments wherein said immune suppressive agent is budesonide.

Preferred methods include embodiments wherein said immune suppressive agent is prednisolone.

Preferred methods include embodiments wherein said immune suppressive agent is tofacitinib.

Preferred methods include embodiments wherein said immune suppressive agent is cyclosporine.

Preferred methods include embodiments wherein said immune suppressive agent is tacrolimus.

Preferred methods include embodiments wherein said immune suppressive agent is everolimus.

Preferred methods include embodiments wherein said immune suppressive agent is azathioprine.

Preferred methods include embodiments wherein said immune suppressive agent is leflunomide.

Preferred methods include embodiments wherein said immune suppressive agent is Mycophenolate.

Preferred methods include embodiments wherein said immune suppressive agent is adalimumab.

Preferred methods include embodiments wherein said immune suppressive agent is anakinra.

Preferred methods include embodiments wherein said immune suppressive agent is certolizumab.

Preferred methods include embodiments wherein said immune suppressive agent is etanercept.

Preferred methods include embodiments wherein said immune suppressive agent is golimumab.

Preferred methods include embodiments wherein said immune suppressive agent is infliximab.

Preferred methods include embodiments wherein said immune suppressive agent is ixekizumab.

Preferred methods include embodiments wherein said immune suppressive agent is natalizumab.

Preferred methods include embodiments wherein said immune suppressive agent is rituximab.

Preferred methods include embodiments wherein said immune suppressive agent is secukinumab.

Preferred methods include embodiments wherein said immune suppressive agent is tocilizumab.

Preferred methods include embodiments wherein said immune suppressive agent is ustekinumab.

Preferred methods include embodiments wherein said immune suppressive agent is vedolizumab.

Preferred methods include embodiments wherein said immune suppressive agent is basiliximab.

Preferred methods include embodiments wherein said immune suppressive agent is daclizumab.

### DETAILED DESCRIPTION OF THE INVENTION

The invention provides means of treating degenerative conditions through the utilization of stem cells that have been modified ex vivo, in vitro or in vivo to possess enhanced potency. In some embodiments the invention provides reduction of oxidative stress prior to/concurrent with, and/or subsequent to stem cell therapy. In other embodiments the invention provides reduction of inflammatory stimuli prior to/concurrent with, and/or subsequent to stem cell therapy. In other embodiments the invention provides normalization of hormonal levels prior to/concurrent with, and/or subsequent to stem cell therapy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the word “a” or “plurality” before a noun represents one or more of the particular noun. For example, the phrase “a mammalian cell” represents “one or more mammalian cells.”

As used herein, the terms “subject” and “patient” are used interchangeably. A patient or a subject can be, for example and without limitation, a human subject, a racehorse or other mammals such as a companion animal (for example, a dog, a cat, etc.). A subject is any mammal that may benefit from the disclosed methods and compositions.

As used herein, the term “Progenitor” cell refers to a stem cell that is in a further stage of cell differentiation. Progenitor cells are unipotent or oligopotent and can get activated in response to injury and other cues, to initiate repair.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

The term “effective amount” or “a therapeutically effective amount” refers to an amount of an agent that provides a beneficial effect to a patient. The term “effective amount” or “a therapeutically effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease or disorder in a patient, or any other desired alteration of a biological system. A beneficial effect can take the form of an improvement over baseline, i.e., an improvement over a measurement or observation made prior to initiation of therapy according to the method. An effective amount or a therapeutically effective amount can be administered in one or more administrations.

The term “autologous” refers to the use of the stem cells, harvested from the same subject who receives it.

The term “Stem cell” is used to describe cells that in the undifferentiated state go that through replication have the capability of both self-renewal and differentiation into mature specialized cells. In broad terms, there are two types of stem cells, embryonic stem cells (ESC) and adult stem cells (ASC). Human ESC are traditionally isolated from a 50- to 150-cell, 4- to 5-day-old post-fertilization blastocyst. ESC generate every specialized cell in the human body; and while capable of indefinite ex vivo proliferation, exist only transiently in vivo-during embryogenesis. ASC are located in tissues throughout the body and function as a reservoir to replace damaged or aging cells. ASC are restricted in their differentiation to cell lineages of the organ system in which they are located. Stem cells are also referred to as toti-, pluri-, multi-, or unipotent. Totipotent stem cells arise from the morula and are capable of forming all cells essential for the body, including placental trophoblasts. In the next stage of embryonic development the morula becomes a blastocyst. Pluripotent stem cells traditionally arise from the blastocyst stage of development and give rise to every cell in the body, but not placental trophoblasts. Pleuripotent stem cells have also been recovered from amniotic fluid and may be found or generated from other sources such as placenta or from manipulation of ASC. ASC are multipotent stem cells in that their differentiation is limited to the tissue lineage or compartment in which they are located. Unipotent stem cells, also termed transient amplifying cells, are restricted in differentiation into a single specialized cell.

In some embodiments, the present invention relates to a method for increasing the successful activity of stem cells and progenitor cells, for example, hematopoietic progenitor/stem cells (HSCs), mesenchymal progenitor/stem cells, mesodermal progenitor/stem cells, endothelial progenitor/stem cells, or ectodermal or neural progenitor/stem cells in vivo in a mammalian subject comprising interacting one or more Wnt/.beta.-catenin signal-, one or more antioxidants, Notch signal- or Hedgehog signal-promoting agents with the progenitor/stem cells in the mammalian subject and increasing the progenitor/stem cells in the mammalian subject. The progenitor/stem cells can include, but are not limited to, hematopoietic progenitor/stem cells (HSCs), stem cells, mesenchymal progenitor/stem cells, mesodermal progenitor/stem cells, endothelial progenitor/stem cells, ectodermal progenitor/stem cells, muscle progenitor/stem cells, endodermal progenitor/stem cells or neural progenitor/stem cells The interacting of one or more Wnt/.beta.-catenin signal-, Notch signal- or Hedgehog signal-promoting agents with progenitor/stem cells, e.g., hematopoietic progenitor/stem cells, occurs either by direct interaction of the signal promoting agents with the hematopoietic progenitor/stem cells or through an indirect interaction between a second signaling factor or cell type acting as an intermediate between the Wnt/.beta.-catenin signal-, one or more antioxidants, Notch signal- or Hedgehog signal-promoting agents and the hematopoietic progenitor/stem cells. Whether the effect on the HSC, or progenitor/stem cell, is on progenitor/stem cell proliferation, survival, cell differentiation, or engraftment into the target tissue, the net effect of activating the Wnt/.beta.-catenin signal is an increase in the measured stem cell/progenitor cell activity. A progenitor/stem cell, e.g., an hematopoietic progenitor/stem cell, can be derived from a variety of sources, including, but not limited to, adult bone marrow, umbilical cord blood cells, or embryonic stem cells, from a mammal, e.g., a human. A method of treating immune related disease is provided comprising administering one or more Wnt/.beta.-catenin signal-, Notch signal- or Hedgehog signal-promoting agents to a mammalian subject and interacting the agent with the hematopoietic progenitor/stem cells of the mammalian subject. A method of treating degenerative disease is provided comprising administering one or more Wnt/.beta.-catenin signal-, Notch signal- or Hedgehog signal-promoting agents to a mammalian subject and interacting the agent with hematopoietic progenitor/stem cells, stem cells, mesenchymal progenitor cells, mesodermal progenitor cells, muscle progenitor cells, endothelial progenitor cells, or neural progenitor cells of the mammalian subject. The degenerative disease includes, but is not limited to, mesenchymal degenerative disease, mesodermal degenerative disease, muscle degenerative disease, endothelial degenerative disease, or neurodegenerative disease.

For “Increasing hematopoietic stem cells in vivo in a mammalian subject” or “increasing hematopoietic stem cells in a mammalian subject” or “increasing the successful activity of progenitor/stem cells, or hematopoietic progenitor/stem cells in vivo in a mammalian subject” or “increasing successful activity of the HSC or progenitor/stem cell” refers to increasing an aspect of the life cycle of the progenitor/stem cell or the hematopoietic progenitor/stem cell (HSC), for example, as a result of a process, including but not limited to, cell proliferation, cell homing to the desired target tissue (e.g., transplanted HSCs are provided intravenously and become established in the bone marrow), decreased apoptosis, self renewal, or increased cell survival. An increase in hematopoietic progenitor/stem cells in vivo can be measured by a cellular assay as disclosed herein (e.g., in vivo hematopoietic stem cell repopulation assay, or hematopoeitic colony-forming unit (CFU) assay) or other cellular assay known in the art. Increased hematopoietic progenitor/stem cells in vivo in a mammalian subject or increased successful activity can be measured by comparing the fold increase in HSCs in a mammalian subject treated by administering one or more Wnt/.beta.-catenin signal-, Notch signal- or Hedgehog signal-promoting agents to the mammalian subject compared to HSCs in a mammalian subject in the absence of such treatment, as measured by any of the cellular activity assays for HSCs or progenitor/stem cells discussed herein or known to one skilled in the art. The baseline number of HSCs in a mammalian subject is considered the successful activity of HSCs in a mammalian subject in the absence of such treatment. The increase in HSCs in the treated mammalian subject by administering one or more Wnt/.beta.-catenin signal-, Notch signal- or Hedgehog signal-promoting agents can be, for example, at least 1.5 fold, at least 2-fold, at least 4-fold, at least 8-fold, or at least 10-fold compared to HSCs in the mammalian subject before treatment. In order to induce “Increasing in vivo mesenchymal progenitor/stem cells or mesodermal progenitor/stem cells in a mammalian subject” or “increasing mesenchymal progenitor/stem cells or mesodermal progenitor/stem cells in a mammalian subject” or “increasing in vivo neural progenitor/stem cells in a mammalian subject” or “increasing neural progenitor/stem cells in a mammalian subject” or “increasing the successful activity of mesenchymal progenitor/stem cells, mesodermal progenitor/stem cells or neural progenitor cells in vivo in a mammalian subject” refers to increasing an aspect of the life cycle of the mesenchymal progenitor/stem cells, mesodermal progenitor/stem cells, neural progenitor cells, muscle progenitor cells or stem cells, for example, as a result of a cellular process, including but not limited to, cell proliferation, cell homing to the desired target tissue (e.g., transplanted stem cells, muscle progenitor cells, or neural progenitor cells are provided intravenously and become established in the bone marrow, muscle, or nerve tissue), decreased apoptosis, self renewal, or increased cell survival. An increase in mesenchymal progenitor/stem cells, mesodermal progenitor/stem cells, neural progenitor cells, muscle progenitor cells or stem cells in vivo can be measured by a cellular assay as disclosed herein (e.g., in vivo hematopoietic stem cell repopulation assay, hematopoeitic colony-forming unit (CFU) assay, in vivo stem cell or progenitor cell repopulation assay, stem cell or progenitor cell colony-forming unit (CFU) assay or other cellular assay known in the art). Increased mesenchymal progenitor/stem cells, mesodermal progenitor/stem cells, neural progenitor cells, muscle progenitor cells or stem cells in vivo in a mammalian subject can be measured by comparing the fold increase in mesenchymal progenitor/stem cells, mesodermal progenitor/stem cells neural progenitor cells, muscle progenitor cells or stem cells in a mammalian subject treated by administering one or more Wnt/.beta.-catenin signal-, Notch signal- or Hedgehog signal-promoting agents to the mammalian subject compared to the number of mesenchymal progenitor/stem cells, mesodermal progenitor/stem cells, neural progenitor cells, muscle progenitor cells or stem cells in a mammalian subject in the absence of such treatment. The increase in mesenchymal progenitor/stem cells, mesodermal progenitor/stem cells neural progenitor cells, muscle progenitor cells or stem cells in the mammalian subject treated by administering one or more Wnt/.beta.-catenin signal-, Notch signal- or Hedgehog signal-promoting agents can be, for example, at least 1.5 fold, at least 2-fold, at least 4-fold, at least 8-fold, or at least 10 fold compared to progenitor/stem cells in the mammalian subject before treatment. Methods for treating disease in a mammalian subject or methods for increasing hematopoietic stem cells in vivo in a mammalian subject are provided by administering a therapeutic composition, for example, a polypeptide, nucleic acid, small molecule, antisense oligonucleotide, ribozyme, RNAi construct, siRNA, shRNA, or antibody, to the mammalian subject. For example, the therapeutic composition can be one or more small molecule modulators of Hedgehog signaling. The therapeutic composition can be a GSK-3 inhibitor. Furthermore, therapeutic efficacy of the Hedgehog pathway antagonist, cyclopamine, has been studied in preclinical models of medulloblastoma, a common malignant brain tumor in children. Berman et al., Science 297: 1559-1561, 2002. Therapeutic efficacy of the Hedgehog pathway agonists have been studied for treatment of traumatic and chronic degenerative conditions. Hedgehog pathway agonists have been shown to target the protein Smoothened. Both antagonists and agonists of the Hedgehog pathway have been shown to target the protein Smoothened. Therapeutic efficacy of a Hedgehog pathway agonist, SAG, a chlorobenzothiophene-containing Hedgehog pathway agonist, binds to Smoothened protein in a manner that antagonizes cylcopamine action. King, Journal of Biology 1:8, 2002; Stecca et al., Journal of Biology 1:9, 2002; Frank-Kamenetsky, et al., Journal of Biology 1:10, 2002; Chen et al., Proc. Natl. Acad. Sci USA 99: 14071-14076, 2002, each incorporated herein by reference in their entirety. ctivation of Notch pathway is preferably achieved by contacting the cell with a Notch ligand, e.g., in soluble form or recombinantly expressed on a cell surface or immobilized on a solid surface, or by introducing into the cell a recombinant nucleic acid expressing a dominant active Notch mutant or an activating Notch ligand, or other molecule that activates the Notch pathway. Agonists of the Notch pathway are able to activate the Notch pathway at the level of protein-protein interaction or protein-DNA interaction. Agonists of Notch include but are not limited to proteins comprising the portions of toporythmic proteins such as Delta or Serrate or Jagged (Lindsell et al., Cell 80: 909-917, 1995) that mediate binding to Notch, and nucleic acids encoding the foregoing (which can be administered to express their encoded products in vivo). and proteins, nucleic acids, small molecules, or derivatives thereof that regulate activity or gene expression of these proteins. In a further embodiment, the agonist is a protein or derivative or fragment thereof comprising a functionally active fragment such as a fragment of a Notch ligand that mediates binding to a Notch protein. In another embodiment, the agonist is a human protein or portion thereof (e.g., human Delta). In another embodiment the agonist is Deltex or Suppressor of Hairless or a nucleic acid encoding the foregoing (which can be administered to express its encoded product in vivo). Human hematopoietic progenitor/stem cells (HSCs) have been identified in bone marrow (BM), peripheral blood and umbilical cord blood (CB). The functional capacity of the cells derived from these tissues can differentiate to a hematopoietic cell fate. In addition, marrow-derived stromal cells were found to differentiate along the osteogenic lineage. Further studies indicated that multipotent mesenchymal progenitor/stem cells (MSCs) reside within the BM, and were capable of giving rise to adipose, bone, cartilage, skeletal muscle and endothelial cell lineages. These combined findings have led to the current notion that BM is therefore a source of both MSCs as well as HSCs. Similar to BM, human HSCs can also be found in umbilical CB and peripheral blood, however, studies aimed at isolating mesenchymal stem/progenitor cells from these alternative hematopoietic sources have provided mixed results. Cells from pre-term CB displayed mesenchymal properties while more recent studies reported a lack of MSCs from full term CB. Similarly, reports have demonstrated the presence or absence of mesenchymal precursors from peripheral blood. Jay et al. Cell Research 14: 268-282, 2004, incorporated herein by reference in its entirety. Human umbilical cord blood (CB) contains a combination of primitive cells and mature cells that have committed to the various hematopoietic lineages. Studies have focused on the characterization and clinical utility of progenitor/stem cells from CB partly due to the ease of obtaining this abundant cell source and the decreased immunogenicity of these cells upon allogenic transplantation. For hematopoietic cell fate, progenitors capable of multi-lineage hematopoiesis reside among cellular subsets of uncommitted CB cells that do not express specific hematopoietic lineage markers. These mature CB cells can be removed based on the surface expression of proteins associated with various hematopoietic lineages to derive a remaining subset of primitive cells referred to as the lineage depleted (Lin.sup.-) fraction. Candidate human HSCs have been shown to exclusively reside in the Lin.sup.- fraction and can be further enriched to Lin.sup.- subsets expressing CD34 but devoid of CD38 (Lin.sup.-CD34.sup.+CD38.sup.-). Subsequent studies identified additional subpopulations of Lin.sup.-cells possessing hematopoietic progenitor function that was devoid of both CD34 and CD38 (Lin.sup.-CD34.sup.-CD38.sup.-), indicating that CD34 may not be unique to the human HSC phenotype. This series of studies illustrates the heterogeneity of the Lin-CD34.sup.- population in human CB and suggests that additional subpopulations may remain to be identified within the Lin-population. A population of cells in human CB devoid of the hematopoietic cell fate marker, CD45 has been identified. Functional analysis of the Lin-CD45-CD34-cells revealed that similar to CD45.sup.-CD34.sup.- cells from BM, these cells possess chondrocytic differentiation potential and hence share properties of mesenchymal progenitors. However, unlike BM-derived mesenchymal progenitor/stem cells, CB derived Lin.sup.-CD45.sup.-CD34.sup.- cells possess unique de novo multi-lineage hematopoietic progenitor capacity. The functional potential displayed by this novel population suggests that Lin.sup.-CD45.sup.-CD34.sup.- cells derived from human CB are potential therapeutic targets for cellular therapies for osteogenic as well as hematopoietic deficiencies and represent a population of human cells with unique developmental potential. Jay et al. Cell Research 14: 268-282, 2004, incorporated herein by reference in its entirety.

In some embodiments of the invention, stem cell activity is enhanced by increasing regulatory T cell activity and/or number. This may be accomplished through several means including administration of low dose interleukin-2. Treatment with IL-2 means that in some embodiments of the invention, stimulation of T regulatory cells in vivo is accomplished by administration of Aldesleukin (Proleukin, Novartis), which is a commercially available IL-2 licensed for the treatment of metastatic renal cell carcinoma in the UK. It is produced by recombinant DNA technology using an Escherichia coli strain, which contains a genetically engineered modification of the human IL-2 gene, and is administered either intravenously or subcutaneously (SC). Following short intravenous infusion, its pharmacokinetic profile is typified by high plasma concentrations, rapid distribution into the extravascular space and a rapid renal clearance. The recommended doses for continuous infusion and subcutaneous injection (as detailed in the Summary of Product Characteristics) are repeated cycles of 18.times.106 IU per m2 per 24 hours for 5 days and repeated doses of 18.times.106 IU, respectively. Peak plasma levels are reached in 2-6 hours after SC administration, with bioavailability of aldesleukin ranging between 31% and 47%. The process of absorption and elimination of subcutaneous aldesleukin is described by a one-compartment model, with a 45 min absorption half-life and an elimination half-life of 3-5 hours. Natural IL-2 was first identified in 1976 as a growth factor for T lymphocytes. It is produced by human cluster designation (CD) 4+ and some CD8+.theta.T-cells and is synthesized mainly by activated T-cells, in particular CD4.sup.+helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilit:ites the proliferation and differentiation of B-cells and the synthesis of immunoglobulin by B-cells, and stimulates the generation, proliferation and activation of natural killer (NK). IL-2 is known to play a central role in the generation of immune responses. In cancer clinical trials, high-dose recombinant IL-2 (e.g., IV bolus dose of 600,000 international units (IU)/kg every 8 hours for up to 14 doses) demonstrated antitumor activity in metastatic renal cell carcinoma (RCC) and metastatic melanoma. Accordingly, such high-dose IL-2 was approved for the treatment of metastatic RCC in Europe in 1989 and in the US in 1992. In 1998, approval was obtained to treat patients with metastatic melanoma. Recombinant human IL-2 (Aldesleukin) (Proleukin.RTM.-Novartis Inc. & Prometheus Labs Inc.) is currently approved by the United States Food and Drug Administration (US FDA). However, IL-2 has a dual function in the immune response in that it not only mediates expansion and activity of effector cells, but also is crucially involved in maintaining peripheral immune tolerance. A major mechanism underlying peripheral self-tolerance is IL-2 induced activation-induced cell death (AICD) in T cells. AICD is a process by which fully activated T cells undergo programmed cell death through engagement of cell surface-expressed death receptors such as CD95 (also known as Fas) or the TNF receptor. When antigen-activated T cells expressing a high-affinity IL-2 receptor (after previous exposure to IL-2) during proliferation are re-stimulated with antigen via the T cell receptor (TCR)/CD 3 complex, the expression of Fas ligand (FasL) and/or tumor necrosis factor (TNF) is induced, making the cells susceptible for Fas-mediated apoptosis. Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control. The particular embodiments discussed below are illustrative only and not intended to be limiting. The ability of T regulatory cells to expand efficacy of BM-MSC in stimulation of angiogenesis, without being bound to theory, occurs through enhancement, in part, of endothelial differentiation. In the context of T regulatory cells, transplanted autologous BM-MNCs in an ischemic area can be incorporated into sites of neovascularization and arranged into the capillary network, whereas transplanted autologous BM-fibroblasts do not participate in the network formation; and direct local transplantation of autologous BM-MNCs, but not of BM-fibroblasts, into ischemic tissue quantitatively and effectively augments neovascularization, collateral vessel formation, and blood flow in the ischemic tissue. In some embodiments, this invention provides a method of forming new blood vessels in tissue in a subject which comprises: a) administration of interleukin-2 locally in a intramuscular manner to enhance numbers and/or activity of T regulatory cells; b) isolating autologous bone marrow-mononuclear cells from the subject; and c) transplanting locally into the tissue an effective amount of the autologous bone-marrow mononuclear cells, thereby forming new blood vessels in the tissue. In one embodiment of this method, the tissue is ischemic tissue, the interleukin-2 is administered in original form or as a depot. In one embodiment, the new blood vessels are capillaries. In a further preferred embodiment of the above-described method the new blood vessels are collateral blood vessels. In another embodiment, both capillaries and collateral blood vessels are formed. It is hypothesized that the transplanted bone-marrow mononuclear cells grow into, i.e. become, the new blood vessels. Through the administration of localized interleukin-2 the teachings of the current disclosure provide a method of increasing blood flow to tissue in a subject which comprises: a) administration of interleukin-2; b) isolating autologous bone-marrow mononuclear cells from the subject; and c) transplanting locally into the tissue an effective amount of the autologous bone-marrow mononuclear cells so as to form new blood vessels in the tissue (angiogenesis), thereby increasing the blood flow to the tissue in the subject.

The disclosure concerns means of augmenting therapeutic activity of regenerative cells for the treatment of degenerative condition, such as regenerative cells that are used at least for anti-inflammatory, angiogenic, regenerative and/or neuroregenerating properties at one or more sites in vivo. In one embodiment of the disclosure, regenerative cells are cultured with cytokines, growth factors, peptides, or combinations thereof prior to administration to an individual, such as a mammal, including humans, horses, dogs, cats, and so forth. In another embodiment, the disclosure encompasses augmentation of regenerative activities for stem cells to be used as therapeutic agents, for example through culture (before and/or during administration to an individual) with one or more agents, such as platelet rich plasma (PRP). In another embodiment the disclosure provides methods for enhancing one or more fibroblast activities for therapeutic activity by co-administering one or more agents and/or PRP, for example together with the regenerative cells such as stem cells. In particular cases the enhanced fibroblasts are delivered to an individual for the purpose of treating a ovarian medical condition in the individual. In some cases an individual is determined to be in need of the enhanced stem cell activity, such as because of degenerative condition or risk thereof. An individual at risk is one that is over the age of about 40, 45, 50, 55, 60, 65, 70, 75, 80, and so forth. In some embodiments, the regenerative cells are exposed to platelet-rich plasma and such exposure directly or indirectly results in enhanced regenerative cells. Numerous growth factors, cytokines and peptides are released from activated platelets, and one approach to therapeutically leverage this is to utilize an autologous platelet concentrate suspended in plasma, also known as platelet-rich plasma (PRP). Several means of preparing PRP are known in the art, some of which are described in the following and incorporated by reference herein [36, 37]. Examples of devices used for generation of PRP include SmartPReP, 3iPCCS, Sequestra, Secquire, CATS, Interpore Cross, Biomet GPS, and Harvest's BMAC [38], for example. Other means of generating PRP are described in U.S. Pat. Nos. 5,585,007; 5,599,558; 5,614,204; 6,214,338; 6,010,627; 5,165,928; 6,303,112; 6,649,072; and 6,649,072, which are incorporated by reference herein in their entirety. In specific embodiments, one can dose PRP at the time of injection in the individual, such as without a prior culture with the fibroblasts. In one embodiment of the disclosure, regenerative cells are delivered systemically or locally to an individual in need thereof, including an individual in need of treatment, including by using a carrier (for example, hydrogel) comprising platelet rich plasma (PRP) and/or hyaluronic acid (HA); in particular cases PRP and/or HA are blended with batroxobin (BTX) as gelling agent. The regenerative cells may be encapsulated in a hydrogel, such as PRP/HA/BTX hydrogel, and cultured, for example in both growing medium and/or medium with or without TGF-01 (for example) for a certain duration of time, such as from one minute (min) up to 21 days. A range of culture duration for any cells may be from 1, 2, 3, 4, 5, 6, 7, 8, 9, 10, 15, 20, 25, 30, 35, 40, 45, 50, 55, 60 (or more minutes or from 1, 2, 3, 4, 5, 6, 7, 8, 9, 10, 11, 12, 13, 14, 15, 16, 17, 18, 19, 20, 21, 22, 23, or 24 or more hours) to 1, 2, 3, 4, 5, 6, 7, 8, 9, 10, 11, 12, 13, 14, 15, 16, 17, 18, 19, 20, or 21 days. The range of time may be from 1, 2, 3, 4, 5, 6, 7, 8, 9, 10, 15, 20, 25, 30, 35, 40, 45, 50, 55, 60 or more minutes to 1, 2, 3, 4, 5, 6, 7, 8, 9, 10, 11, 12, 13, 14, 15, 16, 17, 18, 19, 20, 21, 22, 23, or 24 or more hours. In one embodiment, intra-ovarian administration of stem cells and the hydrogel is performed, which results in jellifies at a certain temperature in a certain period of time. The hydrogel may jellify in 10, 11, 12, 13, 14, 15, 16, 17, 18, 19, or 20 minutes or more at 18, 19, 20, 21, 22, 23, 24, 25, or higher.degree. C. or in 1, 2, 3, 4, 5, or more minutes at 35, 36, 37, 38, 39, or 40 or more.degree. C. in a manner such that the regenerative cells maintain high cell viability and proliferation. In one embodiment the disclosure encompasses the use of fibroblasts for local delivery (such as by intra-disc injections) in individuals with degenerative condition. In such an embodiment, the regenerative cells are cultured in suitable conditions to enhance GAG production, which in at least some cases is achieved by culture with one or more cytokines, such as TGF-beta. Methodologies for growth of mesenchymal stem cells, is incorporated by reference [39].

The disclosure encompasses the use of activation of regenerative cells prior to therapeutic use, including administration of one or more biologically active substances that act as “regenerative adjuvants” for the fibroblasts. The cells in the formulation may display typical regenerative cells morphologies when growing in cultured monolayers. Specifically, cells may display an elongated, fusiform or spindle appearance with slender extensions, or cells may appear as larger, flattened stellate cells that may have cytoplasmic leading edges. A mixture of these morphologies may also be observed. The cells may express one or more proteins characteristic of normal regenerative cells including the fibroblast-specific marker, CD90 (Thy-1), a 35 kDa cell-surface glycoprotein, and the extracellular matrix protein, collagen, as examples. The fibroblast dosage formulation in specific embodiments may be an autologous, allogeneic, or xenogeneic cell therapy product comprising a suspension of regenerative cells, including grown from skin using standard tissue culture procedures as examples.

In certain embodiments, regenerative cells of any kind are utilized in methods for regeneration, and in preparation for (or as part of) these methods, the fibroblasts may be harvested, cultured, and expanded using certain techniques.

Following the obtaining and preparation of regenerative cells prior to delivery to an individual in need thereof, the regenerative cells may be manipulated, including to enhance one or more activities useful for a therapeutic purpose. In some cases, the regenerative cells are exposed to one or more biologically active agents and/or conditions prior to (and/or during) delivery to an individual in need thereof, and in some cases the exposure to one or more biologically active agents and/or conditions prior to (and/or during) delivery may or may not occur during a culturing step. In one embodiment, regenerative cells are pre-activated by contact with a composition or mixture of compositions comprising a biologically active agent that is at least one growth factor, and the growth factor(s) may be selected from the group consisting of transforming growth factors (TGF), fibroblast growth factors (FGF), platelet-derived growth factors (PDGF), epidermal growth factors (EGF), vascular endothelial growth factors (VEGF), insulin-like growth factors (IGF), platelet-derived endothelial growth factors (PDEGF), platelet-derived angiogenesis factors (PDAF), platelet factors 4 (PF-4), hepatocyte growth factors (HGF) and a combination thereof. In certain cases, the growth factors are transforming growth factors (TGF), platelet-derived growth factors (PDGF) fibroblast growth factors (FGF) and a combination thereof. In specific cases, the growth factors are selected from the group consisting of transforming growth factors beta (TGF-beta), platelet-derived growth factors BB (PDGF-BB), basic fibroblast growth factors (bFGF) and a combination thereof. In another embodiment of the disclosure, the growth factor-comprising compositions are delivered to an individual simultaneously with, or subsequent to, delivery of regenerative cells. The delivery may occur by injection, in certain embodiments. The regenerative cells may be autologous, allogeneic, or xenogeneic with respect to the recipient individual. In some embodiments a platelet plasma composition is administered together with the regenerative cells or subsequent to administration of the fibroblasts, and the platelet plasma composition may comprise, consist essentially of, or consist of platelets and plasma and may be derived from bone marrow and/or peripheral blood. The present disclosure may use platelet plasma composition(s) from either or both of these sources, and either platelet plasma composition may be used to regenerate either a nucleus or annulus or both in need thereof. Further, the platelet plasma composition may be used with or without concentrated bone marrow (BMAC). By way of example, when inserted into the annulus, 0.05-2.0 cc of platelet plasma composition may be used, and when inserted into the nucleus, 0.05-3.0 cc of the platelet plasma composition may be used. Platelets are non-nucleated blood cells that as noted above may be found in bone marrow and peripheral blood. In various embodiments of the present disclosure, a platelet plasma composition may be obtained by sequestering platelets from whole blood and/or bone marrow through centrifugation, for example into three strata: (1) platelet rich plasma; (2) platelet poor plasma; and (3) fibrinogen. When using platelets from one of the strata, e.g., the platelet rich plasma (PRP) from blood, one may use the platelets whole or their contents may be extracted and concentrated into a platelet lysate through a cell membrane lysis procedure using thrombin and/or calcium chloride, for example. When choosing whether to use the platelets whole or as a lysate, one may consider the rate at which one desires regeneration and/or tissue healing (which may include the formation of scar tissue without regeneration or healing of a herniated or torn disc). In some embodiments the lysate will act more rapidly than the PRP (or platelet poor plasma from bone marrow). Notably, platelet poor plasma that is derived from bone marrow has a greater platelet concentration than platelet rich plasma from blood, also known as platelet poor/rich plasma, (“PP/RP” or “PPP”). PP/RP or PPP may be used to refer to platelet poor plasma derived from bone marrow, and in some embodiments, preferably PP/RP is used or PRP is used as part of the composition for tissue regeneration. (By convention, the abbreviation PRP refers only to compositions derived from peripheral blood and PPP (or PP/RP) refers to compositions derived from bone marrow

In some embodiments in which the lysate is used, the cytokine(s) may be concentrated in order to optimize their functional capacity. Concentration may be accomplished in two steps. First, blood may be obtained and concentrated to a volume that is 5-15% of what it was before concentration. Devices that may be used include but are not limited to a hemofilter or a hemoconcentrator. For example, 60 cc of blood may be concentrated down to 6 cc. Next, the concentrated blood may be filtered to remove water. This filtering step may reduce the volume further to 33%-67% (e.g., approximately 50%) of what it was prior to filtration. Thus, by way of example for a concentration product of 6 cc, one may filter out water so that one obtains a product of approximately 3 cc. When the platelet rich plasma, platelet poor plasma and fibrinogen are obtained from blood, they may for example be obtained by drawing 20-500 cc of peripheral blood, 40-250 cc of peripheral blood or 60-100 cc of peripheral blood. The amount of blood that one should draw will depend on the number of discs that have degenerated and the size of the discs. As persons of ordinary skill in the art will appreciate, a typical disc has a volume of 2-5 cc or 3-4 cc. In one specific embodiment regenerative cells are treated, or administered together with activated PRP. The method of generation of activated PRP may be used according to U.S. Pat. No. 9,011,929, which is incorporated by reference herein in its entirety and describes essentially: separating the PRP from whole blood, wherein the separating step further comprises the steps of: collecting 10 ml of the whole blood from an animal or patient into a vacuum test tube containing 3.2% sodium citrate, and primarily centrifuging the collected whole blood at 1,750-1,900 g for 3 to 5 minutes; collecting a supernatant liquid comprising a plasma layer with a buffy coat obtained from said centrifugation; transferring the collected supernatant liquid to a new vacuum test tube by a blunt needle, and secondarily centrifuging the collected supernatant liquid at 4,500-5,000 g for 4 to 6 minutes; and collecting the PRP concentrated in a bottom layer by another blunt needle; mixing 1 mL of the PRP collected from the separating step with a calcium chloride solution with a concentration of 0.30-0.55 mg/mL by a three-way connector; and mixing a mixture of the PRP and the calcium chloride solution with type I collagen, wherein the mixing step of mixing the mixture of the PRP and the calcium chloride solution with the type I collagen further comprises the steps of: leaving the type I collagen at a room temperature for 15 to 30 minutes before mixing; and mixing the mixture of the PRP and the calcium chloride solution with the type I collagen with a concentration of 20-50 mg/mL, in an opaque phase, four times by another three-way connector. In some embodiments administration of fibroblasts is performed together with biocompatible polymers and growth factors or PRP, or Platelet Gel.

Treatment of individuals with degenerative condition may be accomplished through one embodiment of the disclosure, such as through the administration of regenerative cells cells that have been genetically modified to upregulate expression of angiogenic stimuli or anti-inflammatory activities. It is known in the art that genes may be introduced by a wide range of approaches including adenoviral, adeno-associated, retroviral, alpha-viral, lentiviral, Kunjin virus, or HSV vectors, liposomal, nano-particle mediated as well as electroporation and Sleeping Beauty transposons. Genes with angiogenic stimulatory function that may be transfected include but are not limited to: VEGF, FGF-1, FGF-2, FGF-4, EGF, HGF, and a combination thereof. Additionally, transcription factors that are associated with upregulating expression of angiogenic cascades may also be transfected into cells used for treatment of lower back pain, including: HIF-lalpha, HIF-2, NET, NF-kB, or a combination thereof. Genes inhibitory to inflammation may be used such as: TGF-a, TGF-b, IL-4, IL-10, IL-13, IL-20 thrombospondin, or a combination thereof, for example. Transfection may also be utilized for administration of genetic manipulation means in a manner to substantially block transcription or translation of genes which inhibit angiogenesis. Antisense oligonucleotides, ribozymes or short interfering RNA may be transfected into cells for use for treatment of lower back pain in order to block expression of antiangiogenic proteins such as: canstatin, IP-10, kringle 1-5, and collagen XVIII/endostatin, for example. Additionally, gene inhibitory technologies may be used for blocking ability of cells to be used for treatment of lower back pain to express inflammatory proteins including: IL-1, TNF-alpha, IL-2, IL-6, IL-8, IL-9, IL-11, IL-12, IL-15, IL-17, IL-18, IL-21, IL-23, IL-27, IFN-alpha, IFN-beta, and IFN-gamma. Globally acting transcription factors associated with inflammation may also be substantially blocked using not only the genetic means described but also decoy oligonucleotides. Suitable transcription factors for blocking include various subunits of the NF-kB complex such as p55, and/or p60, STAT family members, particularly STAT1, STAT5, STAT4, and members of the Interferon Regulatory Factor family such as IRF-1, IFR-3, and IFR-8, for example. Enhancement of angiogenic stimulation ability of the cells useful for the treatment of back pain can be performed through culturing under conditions of restricted oxygen. It is known in the art that stem cells in general, and ones with angiogenesis promoting activity specifically, tend to reside in hypoxia niches of the bone marrow. When stem cells differentiate into more mature progeny, they progressively migrate to areas of the bone marrow with higher oxygen tension.[40]. This important variable in tissue culture was used in studies that demonstrated superior expansion of human CD34 stem cells capable of full hematopoietic reconstitution were obtained in hypoxic conditions using oxygen tension as low as 1.5%. The potent expansion under hypoxia, which was 5.8-fold higher as compared to normal oxygen tension was attributed to hypoxia induction of HIF-1 dependent growth factors such as VEGF, which are potent angiogenic stimuli when released under controlled conditions [41]. Accordingly, culture of cells to be used for treatment of back pain may be performed in conditions of oxygen ranging from 0.5% to 4%, such as 1%-3% and including from 1.5%-1.9%. Hypoxia culture is not limited towards lowering oxygen tension but may also include administration of molecules that inhibit oxygen uptake or compete with oxygen uptake during the tissue culture process. Additionally, in an embodiment of the disclosure, hypoxia is induced through induction of one or more agents that cause the upregulation of the HIF-1 transcription factor. In embodiments wherein the regenerative cells are exposed to hypoxia, the oxygen levels may be between 0.1%-5%, 0.1%-4%, 0.1%-3%, 0.1%-2%, 0.1%-1%, 0.1%-0.75%, 0.1%-0.5%, 0.1%-0.25%, 0.2%-5%, 0.2%-4%, 0.2%-3%, 0.2%-2%, 0.2%-1%, 0.2%-0.75%, 0.2%-0.5%, 0.5%-5%, 0.5%-4%, 0.5%-3%, 0.5%-2%, 0.5%-1%, 0.5%-0.75%, 0.75%-5%, 0.75%-4%, 0.75%-3%, 0.75%-2%, 0.75%-1%, 1%-5%, 1%-4%, 1%-3%, 1%-2%, 2%-5%, 2%-4%, 2%-3%, 3%-5%, 3%-4%, or 4%-5%% oxygen, in specific embodiments. The duration of exposure of the cells to hypoxic conditions, including with (but not limited to) these representative levels of oxygen, may be for a duration of 30 minutes (min)-3 days, 30 min-2 days, 30 min-1 day, 30 min-12 hours (hrs), 30 min-8 hrs, 30 min-6 hrs, 30 min-4 hrs, 30 min-2 hrs, 30 min-1 hour (hr), 1 hr-3 days, 1 hr-2 days, 1 hr-1 day, 1-12 hrs, 1-8 hrs, 1-6 hrs, 1-4 hrs, 1-2 hrs, 2 hrs-3 days, 2 hrs-2 days, 2 hrs-1 day, 2 hrs-12 hrs, 2-10 hrs, 2-8 hrs, 2-6 hrs, 2-4 hrs, 2-3 hrs, 6 hrs-3 days, 6 hrs-2 days, 6 hrs-1 day, 6-12 hrs, 6-8 hrs, 8 hrs-3 days, 8 hrs-2 days, 8 hrs-1 day, 8-16 hrs, 8-12 hrs, 8-10 hrs, 12 hrs-3 days, 12 hrs-2 days, 12 hrs-1 day, 12-18 hrs, 12-14 hrs, 1-3 days, or 1-2 days, as examples only.

Assessment of the anti-inflammatory abilities of regenerative cells generated or isolated for potential clinical use may also be performed. Numerous methods are known in the art, for example they may include assessment of the putative anti-inflammatory regenerative cells cells to modulate immunological parameters in vitro. Putative anti-inflammatory regenerative cells cells may be co-cultured at various ratios with an immunological cell. The immunological cell may be stimulated with an activatory stimulus. The ability of the putative anti-inflammatory cell to inhibit, in a dose-dependent manner, production of inflammatory cytokines or to augment production of anti-inflammatory cytokines, may be used as an output system of assessing anti-inflammatory activity. Additional output parameters may include: proliferation, cytotoxic activity, production of inflammatory mediators, or upregulation of surface markers associated with activation. Cytokines assessed may include: IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, TNF, IFN and/or RANKL. Specific immunological cells may be freshly isolated or may be immortalized cell lines. The immunological cells may be: T cells, B cells, monocytes, macrophages, neutrophils, eosinophils, basophils, dendritic cells, natural killer cells, natural killer T cells, gamma delta-T cells, or a combination thereof. The immunological stimuli may include an antibody, a ligand, a protein, or another cells. Examples including: crosslinking antibodies to T cell receptor, to costimulatory molecules such as CD28, to activation associated molecules such as CD69 or to receptors for stimulatory cytokines such as IL-2. Additional examples of inflammatory stimuli may include co-culture with allogeneic stimulator cells such as in mixed lymphocyte reactions, or may include stimulation with a non-specific activator such as a lectin. Specific lectins may include conconavalin-A, phytohemagluttinin, or wheat germ agglutinin. Other non-specific stimulators may be activators of the toll like receptor pathway such as lipopolysaccharide, CpG DNA motifs or bacterial membrane fractions. The methods described in the above two paragraphs are shown only as examples that may be used to determine, before entry into clinical use, whether a cell population generated as described in the present invention is capable of producing the desired angiogenic stimulatory or anti-inflammatory effects. These examples are only provided as guides which one skilled in the art can optimize upon using routine experimentation.

For any embodiments of the disclosure provided herein, cells to be used for treatment of degenerative condition may be cryopreserved for subsequent use, as well as for transportation, in some cases. One skilled in the art knows numerous methods of cellular cryopreservation. Typically, cells may be treated to a cryoprotection process, then stored frozen until needed. Once needed cells require specialized care for revival and washing to clear cryopreservative agents that may have detrimental effects on cellular function. Generally, cryopreservation requires attention be paid to three main concepts, these are: 1) the cryoprotective agent, 2) the control of the freezing rate, and 3) the temperature at which the cells will be stored. Cryoprotective agents are well known to one skilled in the art and can include but are not limited to dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, or choline chloride as described in U.S. Pat. No. 6,461,645 (incorporated by reference herein in its entirety), for example. A method for cryopreservation of cells that is utilized by some skilled artisans comprises DMSO at a concentration not being immediately cytotoxic to cells under conditions which allow it to freely permeate the cell and to protect intracellular organelles; the DMSO combines with water and prevents cellular damage induced from ice crystal formation. Addition of plasma at concentrations between 20-25% by volume can augment the protective effect of DMSO. After addition of DMSO, cells should be kept at temperatures below 4 C, in order to prevent DMSO-mediated damage. Methods of actually inducing the cells in a state of suspended animation involve utilization of various cooling protocols. While cell type, freezing reagent, and concentration of cells are important variables in determining methods of cooling, it is generally accepted that a controlled, steady rate of cooling is optimal. There are numerous devices and apparatuses known in the field that are capable of reducing temperatures of cells for optimal cryopreservations. One such apparatus is the Thermo Electro Cryomed Freezer.TM. manufactured by Thermo Electron Corporation. Cells can also be frozen in CryoCyte.TM. containers as made by Baxter. One example of cryopreservation is as follows: 2.times.10.sup.6 CD34 cells/ml are isolated from cord blood using the Isolex System.TM. as per manufacturer's instructions (Baxter). Cells are incubated in DMEM media with 10% DMSO and 20% plasma. Cooling is performed at 1 Celsius./minute from 0 to −80 Celsius. When cells are needed for use, they are thawed rapidly in a water bath maintained at 37 Celsius water bath and chilled immediately upon thawing. Cells are rapidly washed, either a buffer solution, or a solution containing a growth factor. Purified cells can then be used for expansion if needed. A database of stored cell information (such as donor, cell origination types, cell markers, etc.) can also be prepared, if desired. In certain embodiments, regenerative cells may be derived from tissues comprising skin, heart, blood vessels, bone marrow, skeletal muscle, liver, pancreas, brain, adipose tissue, foreskin, placental, and/or umbilical cord, for example. In specific embodiments, the fibroblasts are placental, fetal, neonatal or adult or mixtures thereof. The number of administrations of cells to an individual will depend upon the factors described herein at least in part and may be optimized using routine methods in the art. In specific embodiments, a single administration is required. In other embodiments, a plurality of administration of cells is required. It should be appreciated that the system is subject to variables, such as the particular need of the individual, which may vary with time and circumstances, the rate of loss of the cellular activity as a result of loss of cells or activity of individual cells, and the like. Therefore, it is expected that each individual could be monitored for the proper dosage, and such practices of monitoring an individual are routine in the art.

In some embodiments, the cells are subjected to one or more media compositions that comprises, consists of, or consists essentially of Roswell Park Memorial Institute (RPMI-1640), Dublecco's Modified Essential Media (DMEM), Eagle's Modified Essential Media (EMEM), Optimem, Iscove's Media, or a combination thereof. In particular cases, the regenerative cells are recombinantly manipulated to encode SSEA3, VEGF, FGF-1, FGF-2, FGF-4, EGF, HGF, HIF-lalpha, HIF-2, NET, NF-kB, TGF-a, TGF-b, IL-4, IL-10, IL-13, IL-20 thrombospondin, canstatin, IP-10, kringle 1-5, collagen XVIII/endostatin, IL-1, TNF-alpha, IL-2, IL-6, IL-8, IL-9, IL-11, IL-12, IL-15, IL-17, IL-18, IL-21, IL-23, IL-27, IFN-alpha, IFN-beta, IFN-gamma, p55, p60, STAT1, STAT5, STAT4, IRF-1, IFR-3, IFR-8, or a combination thereof. In cases wherein recombination technology is employed, one or more types of the fibroblast cells are manipulated to harbor one or more expression vectors that each encode one or more gene products of interest. A recombinant expression vector(s) can be introduced as one or more DNA molecules or constructs, where there may be at least one marker that will allow for selection of host cells that contain the vector(s). The vector(s) can be prepared in conventional ways, wherein the genes and regulatory regions may be isolated, as appropriate, ligated, cloned in an appropriate cloning host, and analyzed by sequencing or other convenient means. Particularly, using PCR, individual fragments including all or portions of a functional unit may be isolated, where in some cases one or more mutations may be introduced using “primer repair”, ligation, in vitro mutagenesis, etc. as appropriate. The vector(s) once completed and demonstrated to have the appropriate sequences may then be introduced into the host cell by any convenient means. The constructs may be integrated and packaged into non-replicating, defective viral genomes like lentivirus, Adenovirus, Adeno-associated virus (AAV), Herpes simplex virus (HSV), or others, including retroviral vectors, for infection or transduction into cells. The vector(s) may include viral sequences for transfection, if desired. Alternatively, the construct may be introduced by fusion, electroporation, biolistics, transfection, lipofection, or the like. The host cells may be grown and expanded in culture before introduction of the vector(s), followed by the appropriate treatment for introduction of the vector(s) and integration of the vector(s). The cells are then expanded and screened by virtue of a marker present in the construct. The tissue is ischemic tissue or damaged tissue, wherein such tissue requires repair, regeneration or vasculogenesis. In some embodiments, angiogenic cytokines are administered together with IL-2, these include but are not limited to, VEGF-A, VEGF-C P1GF, KDR, EGF, HGF, FGF, angiopoietin-1, and cytokines. In additional preferred embodiments, the nucleic acid molecule encodes endothelial nitric oxide synthases eNOS and iNOS, G-CSF, GM-CSF, VEGF, aFGF, SCF (c-kit ligand), bFGF, TNF, heme oxygenase, AKT (serine-threonine kinase), HIF.alpha.(hypoxia inducible factor), Del-1 (developmental embryonic locus-1), NOS (nitric oxide synthase), BMP's (bone morphogenic proteins), SERCA2a (sarcoplasmic reticulum calcium ATPase), .beta..sub.2-adrenergic receptor, SDF-1, MCP-1, other chemokines, interleukins and combinations thereof. In additional preferred embodiments, genes which may be delivered in the autologous BM-MNCs using the methods of the present invention include but are not limited to nucleic acid molecules encoding factor VIII/von Willebrand, factor IX and insulin, NO creating genes such as eNOS and iNOS, plaque fighting genes thrombus deterrent genes, for example. In a preferred embodiment of the methods described herein, the new blood vessels are capillaries. In another preferred embodiment the new blood vessels are collateral blood vessels. In a further embodiment, both capillaries and collateral blood vessels are formed. This invention provides a method of increasing angiogenesis in diseased tissue in a subject which comprises: a) isolating autologous bone-marrow mononuclear cells from the subject; and b) transplanting locally into the diseased tissue an effective amount of the autologous bone-marrow mononuclear cells, thereby increasing angiogenesis and repair in the diseased tissue in the subject. In a preferred embodiment the tissue is ischemic tissue or tissue in need of repair or regeneration.

1. A method of enhancing stem cell activity in the treatment of a degenerative condition though administering at least one or more antioxidants and/or anti-inflammatory agents before, at the same time has, and subsequent to stem cell therapy.

2. The method of claim 1, wherein said degenerative condition is autoimmune.

3. The method of claim 1, wherein said degenerative condition is immunologically mediated.

4. The method of claim 1, wherein said degenerative condition is mediated by injury.

5. The method of claim 1, wherein said degenerative condition is mediated by genetic predisposition.

6. The method of claim 1, wherein said antioxidant is super oxide dismutase.

7. The method of claim 6, wherein said super oxide dismutase is manganese dependent.

8. The method of claim 7, wherein a gene encoding manganese dependent superoxide dismutase is administered locally into an area proximal to the diseased tissue.

9. The method of claim 8, wherein said gene encoding manganese dependent superoxide dismutase is transfected into diseased tissue by means of a viral vector.

10. The method of claim 8, wherein said gene encoding manganese dependent superoxide dismutase is transfected into diseased tissue by means of a hydrodynamic transfection.

11. The method of claim 8, wherein said gene encoding manganese dependent superoxide dismutase is transfected into diseased tissue by means of a mRNA liposomal delivery.

12. The method of claim 8, wherein said gene encoding manganese dependent superoxide dismutase is transfected into diseased tissue by means of naked DNA administration.

13. The method of claim 1, wherein said antioxidant is zinc.

14. The method of claim 1, wherein said antioxidant is vitamin C.

15. The method of claim 1, wherein said antioxidant is vitamin E.

16. The method of claim 1, wherein said antioxidant is selenium.

17. The method of claim 1, wherein said antioxidant is Trolox.

18. The method of claim 1, wherein said antioxidant is hydroxychloroquine.

19. The method of claim 1, wherein said antioxidant is glutathione.

20. The method of claim 1, wherein said antioxidant is carotene.
